(19) United States
(12) Patent Application Publication
(10) Pub. No.: US 2002/0123064 A1
(43) Pub. Date: Sep. 5, 2002

(54) Hybridization reaction method and hybridization device

(76) Inventors: Keiichi Sato, Kanagawa (JP); Toshiki Morita, Kanagawa (JP)

Correspondence Address: Pillsbury Winthrop LLP, Intellectual Property Group, Suite 200, 11682 El Camino Real, San Diego, CA, 92130, US

(21) Appl. No.: 10/077,163

(22) Filed: Feb. 15, 2002

(30) Foreign Application Priority Data
Feb. 20, 2001 (JP) 43306/2001

(51) Int. Cl. C12Q 1/68; C12M 1/34

(52) U.S. Cl. 435/6; 435/287.2

(57) ABSTRACT

The present invention has an objective of providing a hybridization reaction method where any one can easily and steadily set up, and where evaporation of a sample solution can be minimized.

A silicone sheet 6 is placed in a hollow 11 of a tray 4. A cover glass 8 is placed on the sheet 6 according to a guideline 7 marked on the sheet 6. A sample solution 9 is dropped on the cover glass 8, on which a slide glass having probe DNA fixed thereon is placed with the fixed probe DNA facing down. The hollow 11 is filled with water to prevent the sample solution from evaporating. The obtained tray/cap unit 2 is inserted into a case 1 and sealed with a locking unit 3.

This application claims priority to Japanese Application Serial No. 2001-043306, filed Feb. 20, 2001.

### BACKGROUND OF THE INVENTION

The present invention relates to a hybridization reaction method and a hybridization device for determining the presence or absence of a sequence of interest in a sample biopolymer through hybridization reaction between the sample biopolymer and a probe biopolymer.

Conventionally, hybridization methods that use nucleic acids or proteins with known sequences as probes are often employed in order to identify and/or fractionate a molecular in an organism, particularly in order to detect DNA of interest or the presence of genetic DNA. Specifically, a solution containing fluorescence-labeled sample DNA is dropped on a slide glass that has probe DNA fixed thereon. Then, the slide glass is covered with a cover glass to allow hybridization reaction. The sample DNA that binds to the probe DNA stays with the probe DNA. After washing the slide glass, the fluorescent substance labeling the remaining fixed sample DNA is excited with excitation light from a light source to detect the emitted fluorescence, thereby detecting the hybridized sample DNA.

As a preferable hybridization device for the above-described hybridization reaction, a cassette used for a hybridization reaction thermostat CHBIO is known (Hitachi Software Engineering Co. Ltd.).

FIG. 3 is a perspective view showing a structure of a conventional hybridization device. This hybridization device mainly consists of a case 1 and a tray/cap unit 2 (a housing and a housed member). The tray/cap unit 2 is provided with a tray 4 for holding a slide glass and a packing 5 made of silicone rubber or the like for enhancing sealing. The case 1 and the tray/cap unit 2 can be united with a locking unit 3, whereby the case 1 and the tray/cap unit 2 enclose a sealed space together.

FIG. 4 is a perspective view showing the appearance of the united hybridization device. In the figure, the same reference numerals denote the same components as FIG. 3. The actual dimensions of the united device is 94×41×13 mm³.

The cover glass is used to carry out efficient hybridization reaction with a less amount of sample solution. However, since the cover glass is extremely thin and light, placing and fixing the cover glass on the sample solution at a predetermined position of the slide glass requires certain experience and technique. In the worst case, the glass cover may be broken and the precious sample may be wasted.

Since the amount of the sample solution is small as described above while the hybridization reaction takes place at a relatively high temperature, the sample solution may evaporate. As a result, the sample DNA that remained after the evaporation of the sample solution is hard to wash off and likely to stay on the slide glass, resulting as noise upon analyzing the fluorescent substance.

In view of the above-described problems, the present invention has an objective of providing a hybridization reaction method and a hybridization device which anyone can easily and steadily set up and where the amount of an evaporated sample solution is minimized.

### SUMMARY OF THE INVENTION

Thus, the hybridization reaction method of the present invention comprises the steps of: dropping a sample solution containing a sample biopolymer on a cover glass; and placing a slide glass having a probe biopolymer fixed thereon on the cover glass with the fixed probe biopolymer facing down.

The hybridization reaction method of the invention may further comprise a step of placing the cover glass on a silicon sheet prior to the step of dropping the sample solution. Thus, the cover glass can easily and steadily be fixed at a predetermined position due to adhesiveness between silicone and glass.

The hybridization device of the invention comprises: a tray provided with a hollow for placing a slide glass having a biopolymer fixed thereon; a sheet for fixedly placing the cover glass in the hollow; a case for accommodating the tray; and a cap for sealing the tray with the case.

For example, the sheet is made of silicone. Thus, the cover glass can easily and steadily be fixed at a predetermined position due to adhesiveness between silicone and glass.

The longitudinal length of the sheet is generally equal to the length of the hollow so that the sheet is easily placed at a predetermined position of the hollow.

The sheet has a guideline for defining the positioning of the cover glass to give a positional guide to place the cover glass.

The hybridization device of the invention comprises: a tray having a convex in a hollow for placing a slide glass having a biopolymer fixed thereon; a case for accommodating the tray; and a cap for sealing the tray with the case.

The convex has a cover glass positioning groove for determining the position for placing the cover glass. Accordingly, the cover glass can easily and steadily be placed as a predetermined position, i.e., the cover glass positioning groove 14.

### BRIEF DESCRIPTION OF THE DRAWINGS

FIGS. 1A and 1B are perspective and cross-sectional views showing a structure of a hybridization device according to a first embodiment of the present invention, respectively;

FIGS. 2A and 2B are perspective and cross-sectional views showing a structure of a hybridization device according to a second embodiment of the present invention, respectively;

FIG. 3 is a perspective view showing a structure of a conventional hybridization device; and

FIG. 4 is a perspective view showing an appearance of a united hybridization device.

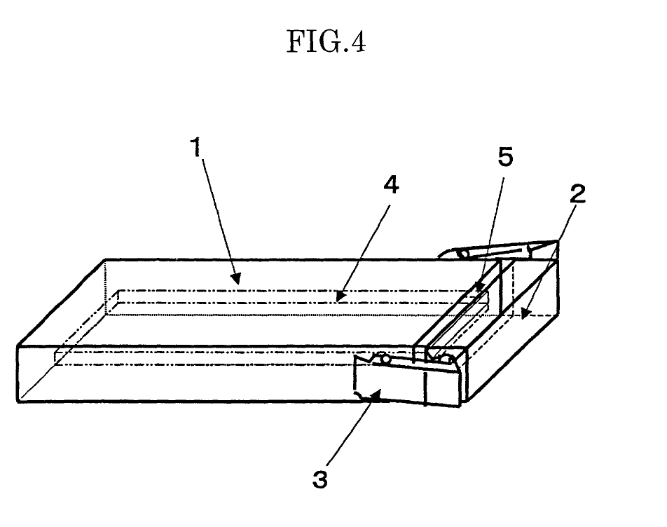

### DETAILED DESCRIPTION OF THE INVENTION

Hereinafter, preferred embodiments of the present invention will be described with reference to the accompanying drawings.

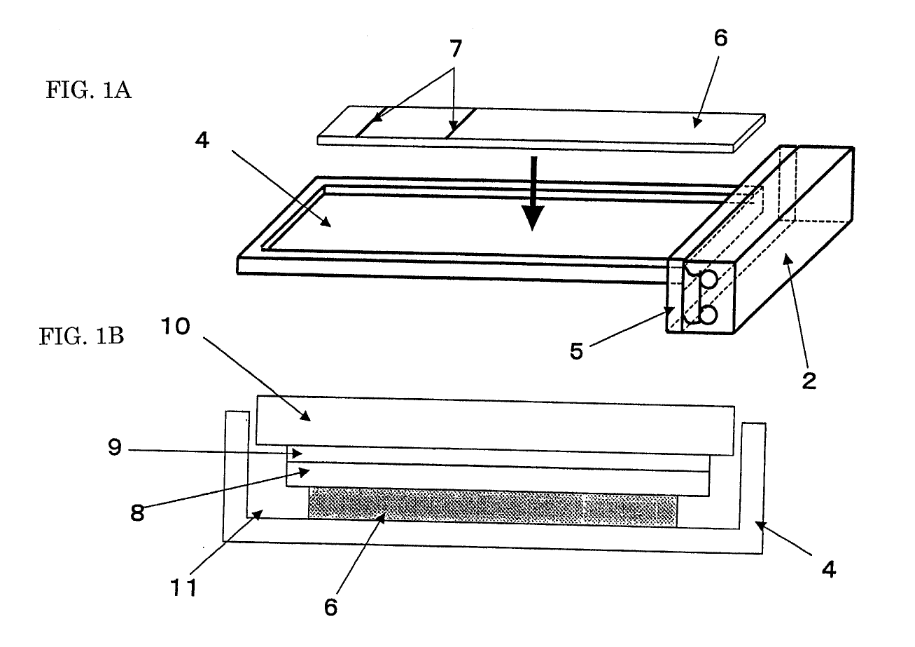

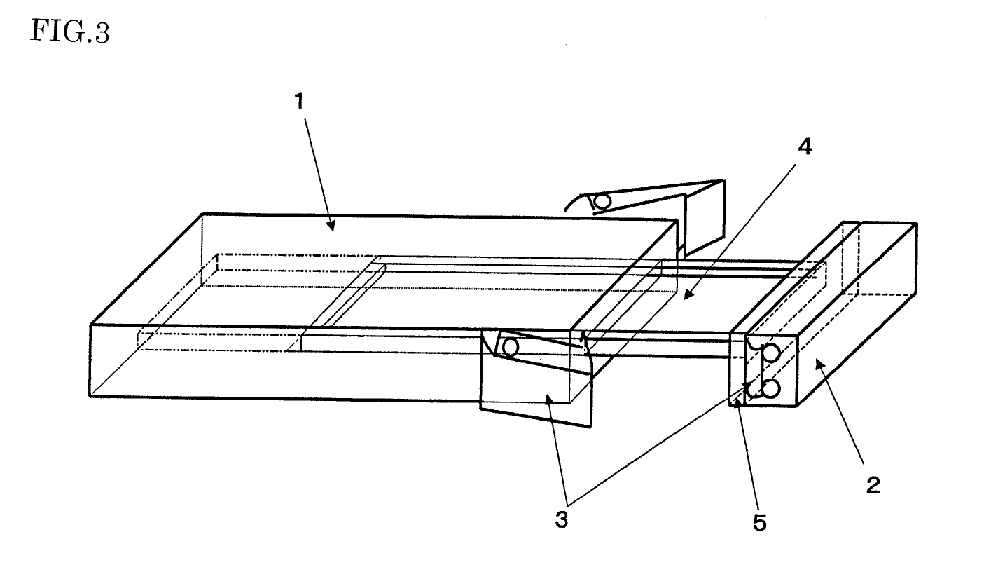

FIGS. 1A and 1B are perspective and cross-sectional views showing a structure of a hybridization device according to Embodiment 1 of the present invention, respectively. Similar to the above-described conventional device, a tray/cap unit 2 of the present embodiment is provided with a tray 4 and a packing 5. According to a hybridization reaction method of the present embodiment, a silicone sheet 6 is placed in a hollow 11 of the tray 4 as shown in FIG. 1B (cross-sectional view of FIG. 1A). A cover glass 8 is placed on the sheet 6 in accordance with the guideline 7 marked on the sheet 6. Then, a sample solution 9 is dropped on the cover glass 8, on which a probe-DNA-fixed slide glass 10 is placed with the probe DNA facing down. The hollow 11 is filled with water to prevent the sample solution from evaporating. This tray/cap unit 2 is inserted into the case 1 (see FIG. 3) and sealed with a locking unit 3. The sealed case 1 is placed under a constant temperature environment to allow hybridization reaction. After the reaction, analysis takes place subsequent to washing, staining and the like.

The tray/cap unit 2 is basically made of an acrylic material and the packing 5 and the sheet 6 are made of silicone. The depth of the hollow 11 is about 3 mm, the thickness of the sheet 6 is about 1 mm and the thickness of the cover glass 8 is about 0.17 mm. The dimensions of the slide glass is 76×26 mm² according to the Japanese standard, 3×1 inch² (25.4 mm²) according to the American standard, and 25×75 mm² according to the European standard. The width of the sheet 6 is shorter than that of the cover glass 8 so that the cover glass 8 can easily be placed and that a greater amount of water for preventing evaporation can be put in.

### ADVANTAGES OF EMBODIMENT 1

(1) In order to arrange the cover glass 8 and the slide glass 10 with the sample solution 9 therebetween, the slide glass 10 of a larger size is mounted on the small cover glass 8 that has been fixedly placed in advance. Thus, only the larger slide glass 10 needs to be handled, which is much easier than handling the smaller cover glass 8.

(2) The longitudinal length of the sheet 6 is generally equal to the longitudinal length of the hollow 11. Thus, the sheet 6 can easily be fixed at a predetermined position of the hollow 11.

(3) The silicone sheet 6 has well adhesiveness with glass. Thus, the cover glass 8 can easily and steadily be placed at a predetermined position of the sheet 6, i.e., a position defined by the guideline 7.

(4) Even if the extremely thin cover glass 8 is broken upon being placed on the sheet 6, the sample solution 9 is not yet dropped at that point and thus the precious sample can be saved.

(5) Since the silicone sheet 6 is hydrophobic, the slide glass 10 can be prevented from getting wet with the water in the hollow 11.

(6) The slide glass 10 is placed by first placing one longitudinal side of the slide glass 10 at the edge of the hollow 11, and then gradually descending a finally placing the whole slide glass 10. As a result, bubbles can be prevented from being contained in the sample solution 9.

(7) Since the sheet 6 is placed in the hollow 11, a sufficient amount of water for preventing evaporation can be used while the volume of the air can be small, thereby minimizing evaporation of the sample solution 9.

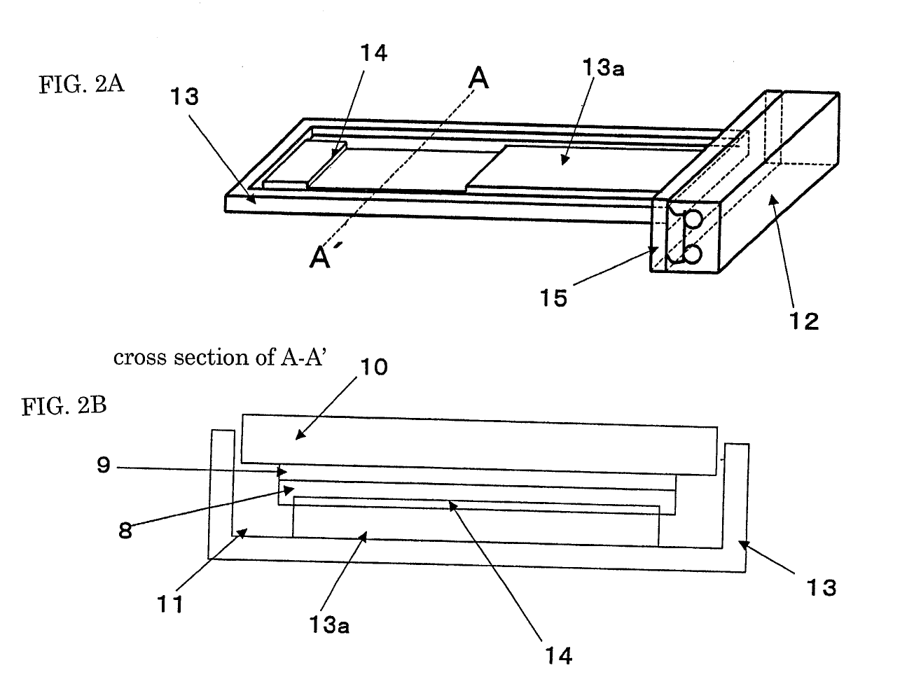

FIGS. 2A and 2B are perspective and cross-sectional views showing a structure of a hybridization device according to Embodiment 2 of the present invention, respectively. Similar to Embodiment 1, a tray/cap unit 12 of the present embodiment has a packing 15 and a sheet/tray unit 13. The sheet/tray unit 13 has a convex 13a in a hollow 11, which serves like the sheet 6 of the Embodiment 1. The convex 13a extends from one end to the other end of the hollow 11 in the longitudinal direction and is provided at the middle in the transversal direction of the hollow 11. According to the present embodiment, the convex 13a (corresponding to the sheet 6 of Embodiment 1) is made of an acrylic material. The convex 13a is provided with a cover glass positioning groove 14 instead of the guideline 7 in Embodiment 1. A hybridization reaction method using the present embodiment is basically the same as Embodiment 1, except that there is no need of placing the sheet 6 and aligning the cover glass 8 with the guideline 7 but instead the cover glass 8 is aligned with the cover glass positioning groove 14.

### ADVANTAGES OF EMBODIMENT 2

(1) In order to arrange the cover glass 8 and the slide glass 10 with the sample solution 9 therebetween, the slide glass 10 of a larger size is mounted on the small cover glass 8 that has been fixedly placed in advance. Thus, only the larger slide glass 10 needs to be handled, which is much easier than handling the smaller cover glass 8.

(2) Since the convex 13a of the sheet/tray unit 13 is provided with the cover glass positioning groove 14, the cover glass 8 can easily and steadily be placed on a predetermined position of the convex 13a, i.e., the cover glass positioning groove 14.

(3) Even if the extremely thin cover glass 8 is broken upon being placed on the sheet/tray unit 13, the sample solution 9 is not yet dropped at that point and thus the precious sample can be saved.

(4) The slide glass 10 is placed by first placing one longitudinal side of the slide glass 10 at the edge of the hollow 11, and then gradually descending and finally placing the whole slide glass 10. Thus, bubbles can be prevented from being contained in the sample solution 9.

(5) Since the convex 13a protrudes from the sheet/tray unit 13 into the hollow 11, a sufficient amount of water for preventing evaporation can be used while the volume of the air can be small, thereby minimizing evaporation of the sample solution 9.

(6) Since the sheet/tray unit 13 saves the use of sheet 6, the number of components used in Embodiment 2 is reduced as compared to the number of components used in Embodiment 1.

The present invention is not limited to the above-described embodiments.

For example, the sheet is not limited to a silicone sheet. Preferably, the sheet has adhesiveness with glass, elasticity, hydrophobic property and no biochemical activity.

The cover glass is not limited to glass and may be made of plastic. Preferably, the cover glass has no biochemical activity.

According to the present invention, the slide glass and the cover glass can be arranged without any special technique. In addition, according to the present invention, evaporation of the sample solution can be minimized.

What is claimed is:

1. A hybridization reaction method, comprising the steps of:
dropping a sample solution containing a sample biopolymer on a cover glass; and
placing a slide glass having a probe biopolymer fixed thereon on the cover glass with the fixed probe biopolymer facing down.

2. A hybridization reaction method according to claim 1, further comprising a step of placing the cover glass on a silicon sheet prior to the step of dropping the sample solution.

3. A hybridization device, comprising:
a tray provided with a hollow for placing a slide glass having a biopolymer fixed thereon;
a sheet for fixedly placing the cover glass in the hollow;
a case for accommodating the tray; and
a cap for sealing the tray with the case.

4. A hybridization device according to claim 3, wherein the sheet is made of silicone.

5. A hybridization device according to claim 3 , wherein the longitudinal length of the sheet is generally equal to the length of the hollow.

6. A hybridization device according to claim 3, wherein the sheet is made of silicone, and the longitudinal length of the sheet is generally equal to the length of the hollow.

7. A hybridization device according to claim 3, wherein the sheet has a guideline for defining the positioning of the cover glass.

8. A hybridization device according to claim 3, wherein the sheet is made of silicone, and the sheet has a guideline for defining the positioning of the cover glass.

9. A hybridization device according to claim 3 , wherein the longitudinal length of the sheet is generally equal to the length of the hollow, and the sheet has a guideline for defining the positioning of the cover glass.

10. A hybridization device, comprising:
a tray having a convex in a hollow for placing a slide glass having a biopolymer fixed thereon;
a case for accommodating the tray; and
a cap for sealing the tray with the case.

11. A hybridization device according to claim 10, wherein the convex has a cover glass positioning groove for determining the position for placing the cover glass.
